(19) United States
(12) Patent Application Publication
(10) Pub. No.: US 2019/0092669 A1
(43) Pub. Date: Mar. 28, 2019

(54) METHOD FOR THE CONTINOUS THERMAL HYDROLYSIS OF ORGANIC MATERIAL AND A PLANT SUITABLE FOR IMPLEMENTING SUCH METHOD

(71) Applicant: AQUATEC, PROYECTOS PARA EL SECTOR DEL AGUA, S.A.U., Madrid (ES)

(72) Inventors: Ivan ARAUZO PEREZ, Castello (ES); Juan PERMUY VILA, Castello (ES)

(73) Assignee: AQUATEC, PROYECTOS PARA EL SECTOR DEL AGUA, S.A.U., Madrid (ES)

(21) Appl. No.: 15/527,083

(22) PCT Filed: Nov. 19, 2015
(86) PCT No.: PCT/ES2015/070825

§ 371 Date: May 16, 2017

(30) Foreign Application Priority Data
Nov. 19, 2014 (ES) P 201431703

(51) Int. Cl. C02F 11/18; C12M 1/00; C02F 1/02

(52) U.S. Cl. CPC C02F 11/18; C02F 1/025; C12M 45/20

(57) ABSTRACT

A method for the continuous thermal hydrolysis of organic material, which includes at least the stages of: a) pressurizing starting organic material at a pressure setpoint P1; b) inline mixing organic material with steam to obtain a mixture at a controlled temperature setpoint T1, selected below the steam saturation temperature of the mixture; c) continuously introducing the mixture obtained, which is in a liquid phase, into a hydrolysis reactor; d) continuously extracting hydrolyzed mixture from the hydrolysis reactor; and e) suddenly depressurizing the hydrolyzed mixture separating a liquid fraction from a steam fraction, which is recovered. The steam employed to produce the mixture in stage b) includes steam recovered in operation e) which is recompressed.

### TECHNICAL FIELD OF THE INVENTION

The invention relates to a method for the continuous thermal hydrolysis of organic material, such as sludge having low solids content, as well as a plant suitable for implementing such method.

### BACKGROUND OF THE INVENTION

The method for the thermal hydrolysis of the sludge produced in wastewater treatment plants is a known process. The thermal hydrolysis process is based on subjecting the sludge to a pressure and temperature state for a given time period and causing a subsequent sudden decompression.

This hydrolyzed sludge presents a rupture of the cell membranes that is why it can be digested more easily by the bacteria during the subsequent digestion process, improving the performance thereof and making it possible to increase the biogas production.

The thermal hydrolysis process is an endothermic process, which requires an energy supply under conditions sufficient to reach both the temperature setpoint and the pressure setpoint that the reaction requires.

The pressure increase is carried out by pumping and has a known low consumption, however the heat energy supplied entails a high operating cost. That is why it is important to optimize the thermal hydrolysis processes at the level of design, in order to minimize the amount of primary energy needed. This improvement can be achieved with methods for recovering the energy dissipated during the sudden decompression and/or use of the energy dissipated with the hydrolyzed sludge. On the other hand, the thermal hydrolysis process carried out continuously favors the decrease of the plant size and consequently the investment cost thereof.

One aim of the present invention is a method by means of which investment costs as well as operative or working costs are minimized.

With regard to operative or working costs, actually there are known proposals essentially aimed to improve the use of energy in the hydrolysis method, which entails a reduction in operational cost

This is the case for example, of the methods providing a sludge preheating stage and in which said sludge and the steam employed to reach the conditions in the reactor are at the same temperature, which means that a liquid (sludge) and a saturated gas (steam) coexist in both, the preheater and the reactor.

On one hand, one of the main disadvantages of this type of processes is that a small decrease in the pressure causes a vaporization of one portion of the liquid phase. For example, in the transfer pumps used with this sludge there are points where the pressure momentarily decreases causing a vaporization and a subsequent collapse as soon as the pressure increases again due to the own pump process. This phenomenon, known as cavitation, decreases the reliability of the pieces of equipment of the plant.

This entails a series of technical problems, which make the implementation of such methods unfeasible at an industrial level, since the pieces of equipment are not prepared for operating at the points and under the working conditions the process demands or would require.

On the other hand, during the sludge preheating stage, the pressure at which this preheating is carried out has to be lower than the pressure of the subsequent sudden decompression, which is carried out in a chamber or flash tank. This fact makes it necessary after the preheating stage to pump the sludge, which as it has been mentioned earlier, is in a saturation state and generates cavitation problems in the equipment.

Therefore, one additional aim of the present invention is a method that can overcome these drawbacks.

### DESCRIPTION OF THE INVENTION

The method for the continuous thermal hydrolysis of organic material according to the invention, which comprises at least the stages of:

a) pressurizing starting organic material at a pressure setpoint P1;

b) inline mixing pressurized organic material with steam to obtain a mixture at a controlled temperature setpoint T1, selected below the steam saturation temperature of the mixture to ensure that the resulting mixture is in a liquid phase;

c) continuously introducing the mixture obtained, which is in a liquid phase, into a hydrolysis reactor;

d) continuously extracting hydrolyzed mixture from the hydrolysis reactor; and

e) suddenly depressurizing said hydrolyzed mixture separating a liquid fraction from a steam fraction, which is recovered;

wherein the steam employed to produce the mixture in stage b) comprises steam recovered in operation e) which is recompressed.

The elimination of one preheating stage and the fact of maintaining the mixture in a liquid phase makes it possible to overcome the drawbacks of known methods.

Herein, liquid phase means that the mixture will not contain steam although it may contain some volatile compounds in smaller amounts, the nature of which will depend on the organic material to be treated.

Advantageously, the method of the invention is a reliable method, given that the state of the current technology makes the pieces of equipment needed for the implementation thereof, work within their normal operating intervals.

Thus, in a preferred variant of the invention the starting organic material is at room temperature.

The method is indicated for keeping the working pressure of the hydrolysis reactor above the steam saturation pressure of the mixture at the temperature at which the process is carried out.

Unlike known methods, the energy recovery is carried out employing steam recompression methods.

In a preferred variant of the method, the starting organic material is at room temperature.

In a variant of the method, the recovered steam is recompressed employing steam recompressing methods, either mechanical, thermal or a combination of both.

According to one embodiment, the steam employed for producing the mixture in stage b) is obtained by mixing new steam with steam recovered from stage e) which is recompressed.

The invention provides for the recovered steam, to be recompressed in a first step, by a steam compressor, which rises the pressure thereof between 4 and 6 bar; and subsequently in a second step, the mechanically recompressed steam is thermally compressed by mixing it with new steam, the differential pressure provided in this second step being between 1 and 1.5 bar.

In a variant of the invention, the inline mixture of pressurized organic material and steam is carried out in a liquid-steam mixing valve.

Preferably, the Temperature setpoint T1, which is comprised between 130 and 190° C., is between 5 and 10° C. lower than the steam saturation temperature of the resulting mixture for pressure P1, which is comprised between 3 and 16 bar.

The stay time of the mixture in the hydrolysis reactor is preferably between 5 and 60 min.

In a variant, stage e) of depressurization is carried out by means of a regulating valve causing a differential pressure ranging between 4 and 8 bar.

In a variant, the liquid fraction obtained in stage e) of depressurization is at a pressure sufficient to pump by pressure difference said liquid fraction to digestion tanks.

According to another aspect of the invention, a plant suitable for implementing the claimed method is disclosed.

This plant, especially suitable for sludges having low solids content, comprises
means for feeding the starting organic material, which comprise a duct and a supply pump that can rise the circulating organic material to an absolute pressure setpoint P1 between 0.3 and 1.6 Mpa (3 and 16 bar);
a mixing device, connected to the feeding means and a steam source, suitable for mixing the pressurized organic material with the steam and obtaining a mixture in liquid state at a controlled temperature setpoint T1, between 130° and 190° C., below the steam saturation temperature of the obtained mixture;
a hydrolysis reactor with at least an inlet and an outlet, the inlet being connected at the outlet of the mixing device and the reactor being prepared to maintain the mixture under absolute pressure conditions ranging between 3 and 16 bar and at a temperature ranging between 130 and 180° C. during the stay thereof inside the reactor;
a regulating valve, connected at the outlet of the hydrolysis reactor, capable of promoting the decompression of the hydrolyzed mixture coming out from the reactor;
an expansion chamber, connected to the regulating valve, where a liquid fraction is separated from a steam fraction, which is recovered;
at least one recompression device of the steam recovered in the expansion chamber; and
at least one mixing device for adding new steam to the recovered and recompressed steam, supplied by the steam source that is connected to the mixing device.

### BRIEF DESCRIPTION OF THE DRAWINGS

FIG. 1, is a schema of a plant according to the invention.

### DETAILED DESCRIPTION OF ONE EMBODIMENT

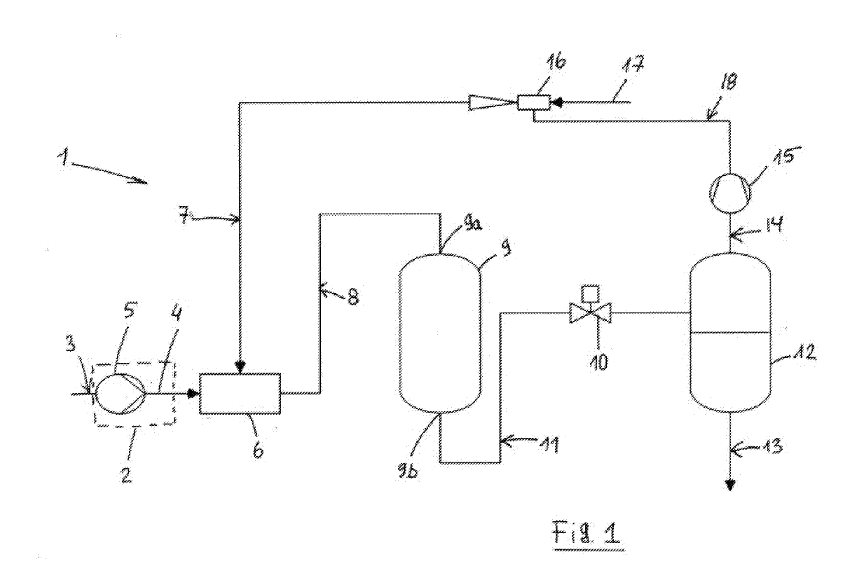

The plant 1 of FIG. 1 is an example of a plant for the continuous thermal hydrolysis of organic material, especially suitable for sludge having low solids content.

With reference to the schema of FIG. 1, the stages of the method and the pieces of equipment included in the exemplary plant for the implementation thereof are indicated below.

The organic material to be hydrolyzed, in the example a starting sludge 3 at room temperature, is fed by means of feeding means 2, which in the example comprise a duct 4 and a supply pump 5 capable of rising the circulating sludge to an absolute pressure setpoint P1 between 3 and 16 bar.

The plant 1 comprises downstream the supply pump 5 a mixing device 6, connected on one side to the feeding means 2 and on the other side to a steam source 7 at a pressure Pv higher or equal to the pressure setpoint P1. The mixing device 6, which may be a liquid-steam mixing valve, mixes the pressurized organic material with steam, thus obtaining a mixture 8, which contains sludge in a heated liquid state.

A characteristic of the method is that said heating is carried out by inline mixing, and controlling that the temperature of the obtained mixture 8 is a controlled temperature setpoint T1 lower than the steam saturation temperature of the mixture, so as to obtain a mixture 8 without steam, essentially in a liquid state. In the example, the Temperature setpoint T1 may be between 5 and 10° C. lower than the saturation temperature for pressure P1, said Temperature setpoint T1 being comprised between 130° and 190° C.

The mixture 8, in liquid phase, is introduced in a hydrolysis reactor 9 through an inlet 9a, provided for such purposes on the upper portion thereof, and is extracted through an outlet 9b, provided in the lower portion of the reactor. In the example, the balance of the material being introduced in and extracted from the reactor 9 is zero.

The working pressure in the reactor 9 is kept above the saturation pressure at the temperature at which the process is carried out, such that the sludge and steam mixture 8 remains in a liquid phase.

The dimensions of the reactor 9 are selected such that the time of stay of the mixture 8 is the appropriate one, preferably being comprised between 5 and 60 min.

Additionally, the reactor 9 is internally provided with a stirring mechanism to prevent eventual problems during the startup and shutdown phases of the plant 1.

A regulating valve 10, connected at the outlet 9b of the hydrolysis reactor 9, brings about the sudden decompression of the hydrolyzed mixture 11 extracted from the reactor 9.

In this stage of sudden decompression, the breaking of the cell walls of the sludge contained in the hydrolyzed mixture 11 takes place. The decompression is carried out using a regulating valve that causes a pressure difference ranging between 4 and 8 bar relative to the pressure setpoint P1, and is confined in an expansion chamber 12, connected to the regulating valve 10, wherein a liquid fraction 13 is separated from a steam fraction 14, which is recovered.

In the example, this recovered steam 14 is subjected in a first step to a mechanical recompression by means of a recompression device 15, in the form of a compressor, the differential pressure provided in this step being between 4 and 6 bar. Then, in a second step the mechanically recompressed steam 18 is thermally compressed by mixing it, in a mixing device 16 based on the Venturi effect, with new steam 17, the differential pressure provided in this second step being between 1 and 1.5 bar, thus obtaining the steam source 7 that feeds the mixing device 6.

1. A method for the continuous thermal hydrolysis of organic material, which comprises at least the stages of:
a) pressurizing starting organic material (3) at a pressure setpoint P1;
b) inline mixing pressurized organic material with steam (7) to obtain a mixture (8) at a controlled temperature setpoint T1, selected below the steam saturation temperature of the mixture to ensure that the mixture is in a liquid phase;
c) continuously introducing the mixture (8) obtained, which is in a liquid phase, in a hydrolysis reactor (9);
d) continuously extracting hydrolyzed mixture (11) from the hydrolysis reactor (9); and
e) suddenly depressurizing said hydrolyzed mixture separating a liquid fraction (13) from a steam fraction, which is recovered (14);
wherein the steam (7) employed to produce the mixture in stage b) comprises steam recovered (14) in operation e) which is recompressed.

2. The method according to claim 1, characterized in that the starting organic material (3) is at room temperature.

3. The method according to claim 1, characterized in that the working pressure of the hydrolysis reactor (9) is kept above the steam saturation pressure at the temperature at which the process is carried out.

4. The method according to claim 1, characterized in that the recovered steam (14) is recompressed employing steam recompressing methods either mechanical, thermal or a combination of both.

5. The method according to claim 1, characterized in that the steam (7) employed for producing the mixture (8) in step b) is obtained by mixing new steam (17) with steam (14) recovered from stage e) which is recompressed.

6. The method according to claim 4, characterized in that the recovered steam (14) is recompressed in a first step, by a steam compressor, which rises the pressure thereof between 4 and 6 bar; and subsequently in a second step the mechanically recompressed steam (18) is thermally compressed by mixing it with new steam (17), the differential pressure provided in this second step being between 1 and 1.5 bar.

7. The method according to claim 5, characterized in that the inline mixture of pressurized organic material and steam is carried out in a liquid-steam mixing valve.

8. The method according to claim 1, characterized in that the Temperature setpoint T1 is between 5 and 10° C. lower than the steam saturation Temperature of the mixture for pressure P1.

9. The method according to claim 1, characterized in that the stay time of the mixture in the hydrolysis reactor (9) is between 5 and 60 min.

10. The method according to claim 1, characterized in that stage e) of depressurization is carried out by means of a regulating valve causing a differential pressure ranging between 4 and 8 bar.

11. The method according to claim 1, characterized in that the liquid fraction (13) obtained in stage e) of depressurization is at a pressure sufficient to pump by pressure difference said liquid fraction (13) to digestion tanks.

12. The method according to claim 1, characterized in that the pressure setpoint P1 is between 3 and 16 bar.

13. The method according to claim 1, characterized in that the Temperature setpoint T1 is between 130 and 190° C.

14. A plant (1) for the continuous thermal hydrolysis of organic material, especially suitable for sludge having low solids content, comprising
means (2) for feeding the starting organic material (3), which comprise a duct (4) and a supply pump (5) capable of rising the circulating organic material to an absolute pressure setpoint P1 between 3 and 16 bar;
a mixing device (6), connected to the feeding means (2) and a steam source (7), suitable for mixing the pressurized organic material with the steam and obtaining a mixture (8) in a liquid state at a controlled temperature setpoint T1, between 130° and 190° C., below the steam saturation temperature of the mixture;
a hydrolysis reactor (9) with at least an inlet (9a) and an outlet (9b), the inlet (9a) being connected at the outlet of the mixing device (6) and the reactor being prepared to maintain the mixture under absolute pressure conditions ranging between 3 and 16 bar and at a temperature ranging between 130 and 180° C. during the stay thereof inside the reactor;
a regulating valve (10), connected at the outlet (9b) of the hydrolysis reactor (9), capable of promoting the decompression of the hydrolyzed mixture (11) coming out from the reactor;
an expansion chamber (12), connected to the regulating valve (10), where a liquid fraction (13) is separated from a steam fraction (14), which is recovered;
at least one recompression device (15) of the steam (14) recovered in the expansion chamber (12); and
at least one mixing device (16) for adding new steam (17) to the recovered and recompressed steam (18), supplied by the steam source (7) connected to the mixing device (6).
